(19) United States
(12) Patent Application Publication
(10) Pub. No.: US 2023/0132952 A1
(43) Pub. Date: May 4, 2023

(54) PHOTOBIOMODULATION DEVICE

(71) Applicants: Jimmy Youngblood, Richmond, TX (US); Matthew Currier, Cypress, TX (US)

(72) Inventors: Jimmy Youngblood, Richmond, TX (US); Matthew Currier, Cypress, TX (US)

(21) Appl. No.: 17/979,466

(22) Filed: Nov. 2, 2022

Related U.S. Application Data
(60) Provisional application No. 63/275,304, filed on Nov. 3, 2021.

(51) Int. Cl. A61N 5/06; G16H 40/63; G16H 20/40

(52) U.S. Cl. CPC A61N 5/06; G16H 40/63; G16H 20/40; A61N 2005/0627; A61N 2005/0652

(57) ABSTRACT

A photobiomodulation device and a method of using a photobiomodulation device are described. The photobiomodulation device includes a memory, a light emitting array, and a controller coupled to the memory and the light emitting array. In the photobiomodulation device, the controller is configured to cause a plurality of light sources of the light emitting array to respectively emit a first changing spectrum of light according to a set of instructions stored in the memory. According to the method the photobiomodulation device causes a plurality of light sources of a light emitting array of the photobiomodulation device to respectively emit a first changing spectrum of light according to a set of instructions.

### PRIORITY CLAIM

This application claims priority to and the benefit of United States Provisional Patent Application No. 63/275,304, titled Photobiomodulation Embedded Controller, and filed on Nov. 3, 2021, at the United States Patent and Trademark Office, the entire content of which is incorporated by reference herein as if fully set forth below in its entirety for all applicable purposes.

### FIELD

The present disclosure generally relates to photobiomodulation (PBM) and, more particularly, to photobiomodulation devices and operating methods related to photobiomodulation devices.

### BACKGROUND

Photobiomodulation may refer to several therapeutic techniques that employ low-level laser, light emitting diode (LED), or other light-generating devices to relieve pain or heal wounds, for example. Photobiomodulation may also be referred to as phototherapy or PBM therapy. Some devices that apply photobiomodulation techniques to patients may have simplistic manual controls (e.g., on/off switch, timer, wavelength selection, and intensity adjustment) or, if they are automatically controlled, the controls may be preset and not changeable by a user. Some devices may have wireless controls (e.g., a wireless remote control), and some devices may have a mobile application, both of which allow a user to control the photobiomodulation device; however, the wireless remote control and mobile application may only allow a user to actuate the same simplistic manual controls (e.g., on/off switch, timer, wavelength selection, and intensity adjustment) as the user might be able to actuate from a control panel mounted on and/or hardwired to the photobiomodulation device.

### BRIEF SUMMARY OF SOME EXAMPLES

The following presents a simplified summary of one or more aspects of the present disclosure to provide a basic understanding of such aspects. This summary is not an extensive overview of all contemplated features of the disclosure. It is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description presented below.

In one example, a photobiomodulation device is described. The photobiomodulation device includes a memory, a light emitting array, and a controller coupled to the memory and the light emitting array. In the example, the controller is configured to cause a plurality of light sources of the light emitting array to respectively emit a first changing spectrum of light according to a set of instructions stored in the memory.

In another example, a method operational at a photobiomodulation device is described. the method includes causing a plurality of light sources of a light emitting array of the photobiomodulation device to respectively emit a first changing spectrum of light according to a set of instructions.

These and other aspects of the invention will become more fully understood upon a review of the detailed description that follows. Other aspects and features of the present invention will become apparent to those of ordinary skill in the art upon reviewing the following description of specific examples of the present invention in conjunction with the accompanying figures. While features of the present invention may be discussed relative to certain examples and figures below, all features of the present invention can include one or more of the advantageous features discussed herein. In other words, while one or more aspects may be discussed as having certain advantageous features, one or more of such features may also be used in accordance with the various examples of the invention discussed herein.

### BRIEF DESCRIPTION OF THE DRAWINGS

The features, nature, and advantages of the present aspects may become more apparent from the detailed description set forth below when taken in conjunction with the drawings in which like reference characters identify correspondingly throughout.

FIG. 1 is a block diagram of a photobiomodulation device according to some aspects of the disclosure.

FIG. 2 is an isometric front-right view of an exemplary photobiomodulation system according to some aspects of the disclosure.

FIG. 3 is a conceptualized schematic view of a person standing in front of, and receiving photobiomodulation therapy from, an array of photobiomodulation devices, such as the photobiomodulation device of FIG. 2, according to some aspects of the disclosure.

FIG. 4 is a flow chart illustrating one example of an overall process that may be used with a photobiomodulation device according to some aspects of the disclosure.

FIG. 5 is a flow chart depicting a process associated with a photobiomodulation device according to some aspects of the disclosure.

FIG. 6 is a block diagram illustrating an example of a hardware implementation of a photobiomodulation device employing a processing system according to some aspects of the disclosure.

FIG. 7 is a flow chart illustrating an example of a process of photobiomodulation therapy at a photobiomodulation device according to some aspects of the disclosure.

FIG. 8 is a flow chart illustrating an example of a process of updating set of instructions of a user at both a photobiomodulation device and at a server associated with the photobiomodulation device according to some aspects of the disclosure.

FIG. 9 is a flow chart illustrating an example of a process associated with machine learning according to some aspects of the disclosure.

FIG. 10 is a flow chart illustrating an example of a process of modifying parameters associated with an emitting of a spectrum of light in response to changes in physiological data of a user according to some aspects of the disclosure.

### DETAILED DESCRIPTION

In the following description, specific details and examples are given to provide a thorough understanding of the subject matter. However, it will be understood by one of ordinary skill in the art that the examples may be practiced without these specific details.

Photobiomodulation devices that emit light predominate the United States market. The therapy provided by photobiomodulation devices may be referred to as light therapy or photobiomodulation (PBM) therapy. Some photobiomodulation devices may utilize light emitting diodes (LEDs) to emit light. Some photobiomodulation devices may utilize Low Level Lasers to emit light. In some examples, a photobiomodulation device may include two pluralities of lights, with a first plurality of lights often emitting light in a first spectrum and a second plurality of lights often emitting light in a second spectrum, different from or overlapping with the first spectrum. In general, photobiomodulation devices in the United States market today may have a mechanical or an electronic timer that turns the first, or if two pluralities of lights are provided, both the first and the second plurality of LEDs, on for a certain amount of time. Multiple and different length on-off cycles are not supported. Pulse controls are not supported. Brightness controls are not supported. For example, in a photobiomodulation device, a user may set the first plurality of lights to be on for a first specific amount of time, set the second plurality of lights to be on for a second specific amount of time, or set both the first and the second pluralities of lights to be on for a third specific amount of time. Once the specific amount of time allotted to a given plurality of lights expires, that plurality of lights may not be illuminated again (unless and until the user resets the photobiomodulation device).

Some photobiomodulation devices may be “daisy-chained” together using a leader/follower designation where all photobiomodulation devices except one in the circuit are followers, with the remaining one being the leader. The leader may control the on-time and durations of the followers. In such an example, all followers may operate exactly as the leader operates and the leader simply operates as described above (e.g., enables a plurality of lights to be on for a given amount of time).

However, examples of photobiomodulation devices described herein may facilitate the execution of sets of instructions (sometimes referred to as recipes herein) that may configure individual light sources in a first plurality of light sources to respectively emit one or more wavelengths of light, or may configure a second plurality of light sources in the first plurality of light sources (e.g., where the second plurality of light sources may define one channel of light sources) to respectively emit one or more wavelengths of light, or may configure multiple third pluralities of light sources in the first plurality of light sources (e.g., multiple channels of light sources) to respectively emit one or more wavelengths of light. Any of the preceding individual light sources, second plurality of light sources, and/or third pluralities of light sources may be activated at various times for various durations, activated with various intensities, activated according to various patterns of repetition, or any combination thereof. Any set of instructions may be designed to obtain respective health outcomes or may be designed to obtain other outcomes (e.g., related to plant growth, sleep, disinfection, etc.). The set of instructions may also be referred to as a recipe or a protocol herein. According In some examples, a given set of instructions may be altered based on feedback from a user. In some examples, a given set of instructions may be altered based on feedback from various sensors that may be used to monitor a user receiving a photobiomodulation treatment.

The examples of photobiomodulation devices described herein may be used to treat users according to established research outcomes, where such outcomes may be a goal of the treatment. The goal of achieving a certain outcome stands in contrast to a goal of simply applying light of a given wavelength for a predetermined amount of time.

For example, an established research outcome may be the alleviation of a symptom of a given skin condition. Research may indicate that that users with the given skin condition report a reduction of symptoms (e.g., itchiness, splotches, cracked or dry skin, etc.) after being exposed to red light at about 650 nm, near-infrared light at about 800 nm, red light at 700 nm, and near-infrared light at about 850, where each of the first two illuminations at the first two wavelengths occur in series for respective durations of about 5 minutes, followed by a simultaneous illumination of the remaining two wavelengths for about 10 minutes, followed by two repetitions of all four wavelengths according to the sequences and for the durations just described. In general, the light may be applied in series or simultaneously across channels. The previous examples of wavelengths of light are exemplary and not limiting. Any of one or more wavelengths of light (for example, but not limited to, light in the red, near infrared, and ultraviolet) are within the scope of the disclosure.

The established research outcome may have been reached by aggregating and analyzing data from clinical trials that included exposing a plurality of users of a plurality of ages and genders having the given skin condition to various combinations of wavelengths, durations, and patterns of repetition of light. Of course, this is only an example, and other ways to reach established research outcomes are within the scope of the disclosure.

The photobiomodulation devices described herein may be used to administer light (of any wavelength or combination of wavelengths) according to predefined sets of instructions (e.g., recipes, protocols), where the goal of administering the light (e.g., administering photobiomodulation therapy) may be to obtain certain therapeutic outcomes and perform research. Therapeutic outcomes may include, for example, the alleviation of a symptom. By performing research, one may determine which wavelengths of light, and which patterns of application of that light, may be administered to a subject to obtain the alleviation of the symptom. Furthermore, data, in the nature of feedback provided by users of photobiomodulation devices, may be collected as part of the research, and may be used to modify a given set of instructions to improve the outcome perceived by any individual user. The modified sets of instructions (e.g., modified in view of feedback provided by a plurality of users having similar demographic properties) may be provided to new recipients of photobiomodulation therapy. The modified sets of instructions may provide improved outcomes for the new recipients. The photobiomodulation therapy may be provided to a user by a photobiomodulation device by having a processor of a processing system of the photobiomodulation device execute a given set of instructions.

Certain aspects of photobiomodulation use of ultraviolet (UV) light will now be discussed. The addition of ultraviolet light to red light and near infrared light that may be used in photobiomodulation devices utilized for therapeutics (e.g., improvements to health and well-being) may facilitate a use of the photobiomodulation device for additional purposes including the growth of plants and cleaning and sterilization. Plants and human beings may be referred to as biological beings herein.

The use of UV light with photobiomodulation devices is referred to as photobiomodulation-UV (PBM-UV) herein. Incorporating PBM-UV to photobiomodulation device may allow the photobiomodulation device to administer UV light as part of a photobiomodulation therapy and to use the same UV light for cleaning and sterilization after application of the photobiomodulation therapy. According to this aspect, a cleaning protocol that utilizes UV light emitted from the photobiomodulation device may be implemented following a therapeutic photobiomodulation therapy session. While some therapeutic photobiomodulation devices may make use of the “red” end of the electromagnetic spectrum (e.g., about 660 nm and above), there are no known therapeutic photobiomodulation devices that utilize UV light sourced from the photobiomodulation device itself to effectuate cleaning of the photobiomodulation device or the air and surfaces surrounding the photobiomodulation device that may be illuminated by UV light emitted from the photobiomodulation device.

UV light has documented, positive outcomes on biological creatures (e.g., humans, plants, users of a photobiomodulation device, such as those described herein) and their environments; these effects are both direct and indirect. Accordingly, PBM-UV may have two general use cases for PBM-UV; namely, direct, and indirect.

Direct application of UV light (e.g., UV-A, UV-B, etc.) to a biological creature has known associated health effects depending, for example, on the duration, (e.g., duration in terms of on/off time, pulse width, duty cycle, as well as in terms of a number of times to repeat an illumination), wavelength, and intensity of the applied UV light. As described herein, a photobiomodulation device may be configured for direct application of UV light (as well as other wavelengths of light) to a biological creature (e.g., both human and plant), where the application of the light may be controlled by a processing system (e.g., a controller, a processor). According to some aspects, the processing system may be configured to automatically disseminate a dosage of UV light (as well as other wavelengths of light) in accordance with a set of instructions (e.g., a prescribed protocol, a recipe) designed to obtain a specific health outcome and/or designed to disinfect a surface illuminated by the light, and/or designed to obtain other outcomes (e.g., plant growth, improve sleep, etc.). A measure of the disseminated dosage may, in some examples, be defined by a combination of at least two of: an exposure time, exposure intensity, exposure wavelength, or a pattern or cycle of exposure.

Indirect application of UV light may refer to an application of a spread-spectrum of UV light to an unoccupied space for a predetermined amount of time. UV light tends to kill free-standing microorganisms in the air and on surfaces. UV light does not linger after application as do some cleaning solutions. UV light does not harm electronic devices in certain dosages, unlike abrasive and wet cleaning solutions.

PBM-UV incorporates light sources (emitters) that generate light in the spectrum of less than 450 nm. According to some aspects, one model of a photobiomodulation device may incorporate general UV light emission. Another model of a photobiomodulation device emit multiple wavelengths of UV light, such as any combination of one or more of UV-A, UV-B, and UV-C. Still another model of a photobiomodulation device may emit multiple wavelengths of UV light and/or be configured to vary the intensity of one or more of the emitted wavelengths of light.

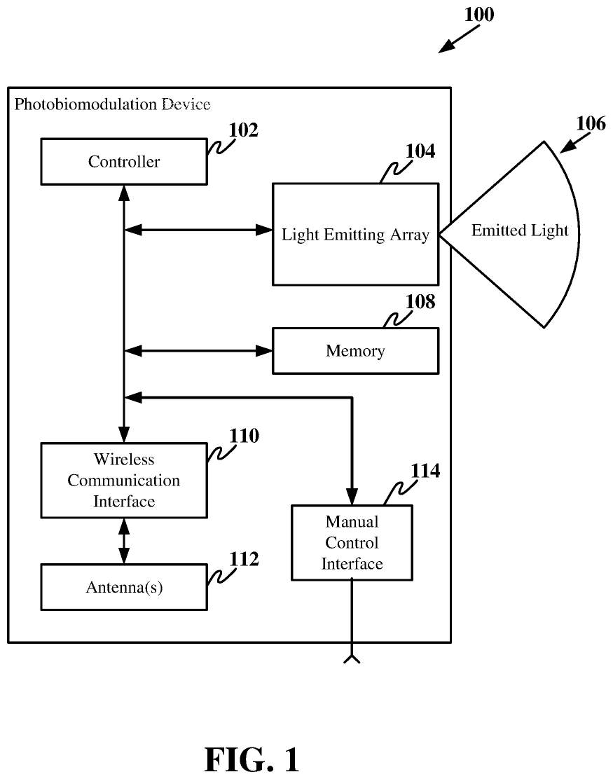

FIG. 1 is a block diagram of a photobiomodulation device 100 according to some aspects of the disclosure. The photobiomodulation device 100 may include a controller 102 (e.g., a processing system) and a light emitting array 104 (e.g., a panel having an array of various combinations of LEDs and/or low level laser emitters). The light emitting array 104 may produce emitted light 106 that may be defined in terms of, for example, wavelength, duration of illumination, intensity of illumination, and pattern of repetition of illumination. The photobiomodulation device 100 may receive power from a power source (not shown). The power source may be and alternating current (AC) or a direct current (DC) power source. For example, the power source may be a 120V, 60 Hz AC power source, a 240V 60 Hz AC power source, or a direct current (DC) power source, such as a 28 V DC power source. The preceding list is exemplary and not limiting. Other AC voltages and frequencies, or other DC voltages are within the scope of the disclosure.

The photobiomodulation device 100 may also include a wireless communication interface 110 and one or more antennas 112. The wireless communication interface 110 may be a wireless transceiver. For example, the wireless communication interface 110 may be configured to permit a user to interface with the photobiomodulation device 100 via Bluetooth®, WiFi®, WiMAX®, LTE, 4G, 5G and beyond, or the like. The one or more antennas 112 may be configured to operate at the frequency or frequencies utilized by the wireless communication interface 110. In some examples, in addition to or instead of the wireless communication interface 110 and the one or more antennas 112, the photobiomodulation device 100 may include a manual control interface 114 to which a wired keyboard, keypad, or gaming controller (not shown) may be coupled. The preceding list is exemplary and non-limiting. Coupling may be via any type of connector, such but not limited to a USB, RJ11, or RJ45 connector.

According to some aspects, the controller 102 of the photobiomodulation device 100 may provide for remote or local control of the photobiomodulation device 100. The controller 102 may accept and may store (e.g., in a memory 108) a plurality of sets of instructions that cause the controller to vary the operating parameters of the light emitting array 104. The controller 102 may control the photobiomodulation device 100 and may also control one or more other photobiomodulation devices that may be similar to or different from the photobiomodulation device 100. Operational parameters may include, but are not limited to, wavelength, duration, intensity, and pattern of repetition of illumination of light emitted by the light emitting array 104. These plurality of sets of instructions may be referred to as a plurality of recipes, prescriptions, or commands herein. Each set of instructions may be configured to alter the emitted light 106 emanating from the light emitting array 104. Parameters that may be controlled using the set of instructions include, but are not limited to, wavelength, duration, intensity, and/or pattern of repetition of illumination. The duration may include an overall duration of a complete therapeutic session as well as individual durations of individual illuminations of various light sources of the light emitting array 104 at various times during the complete therapeutic session. Furthermore, when utilizing a photobiomodulation device for cleaning and/or sterilization as well as for therapeutic purposes, an additional duration during which direct or indirect PBM-UV may be used for cleaning and/or sterilization may be added to the overall duration of the complete therapeutic session.

According to some aspects, the controller 102 may be programmed using open-source code. Open-source code may allow a user to program at least some control aspects of the photobiomodulation device using a programming language that is generally known and useable to persons without a fee. Accordingly, the ability of a user to customize the operation of the controller 102 or the photobiomodulation device 100 may be increased beyond the ability of users of photobiomodulation devices that may use closed source protocols. Closed-source protocols may be proprietary in nature and may utilize programming languages that are generally not known or not useable to persons without a fee, subscription, or license, for example. Control by the controller 102 may permit the emitted light 106 to have a uniform distribution or any distribution across various wavelengths at any given moment of operation of the photobiomodulation device 100.

According to some aspects, the photobiomodulation device 100 may utilize machine learning to select and/or modify a set of instructions associated a particular photobiomodulation therapy and as particular user. The set of instructions may be stored in the memory 108 or may be stored remote from the photobiomodulation device 100, for example stored at a remote server or in cloud storage. The set of instructions may be executed by the controller 102 (e.g., a processing system) of the photobiomodulation device 100. Machine learning, as used herein, may refer to a process by which the controller 102 can learn and adapt without following explicit instructions, for example, by using algorithms and statistical models to analyze and draw inferences from patterns in data. In some examples, the data may be collected by sensors (not shown) that may be coupled to the controller 102 and may supply user biometric data to the controller 102.

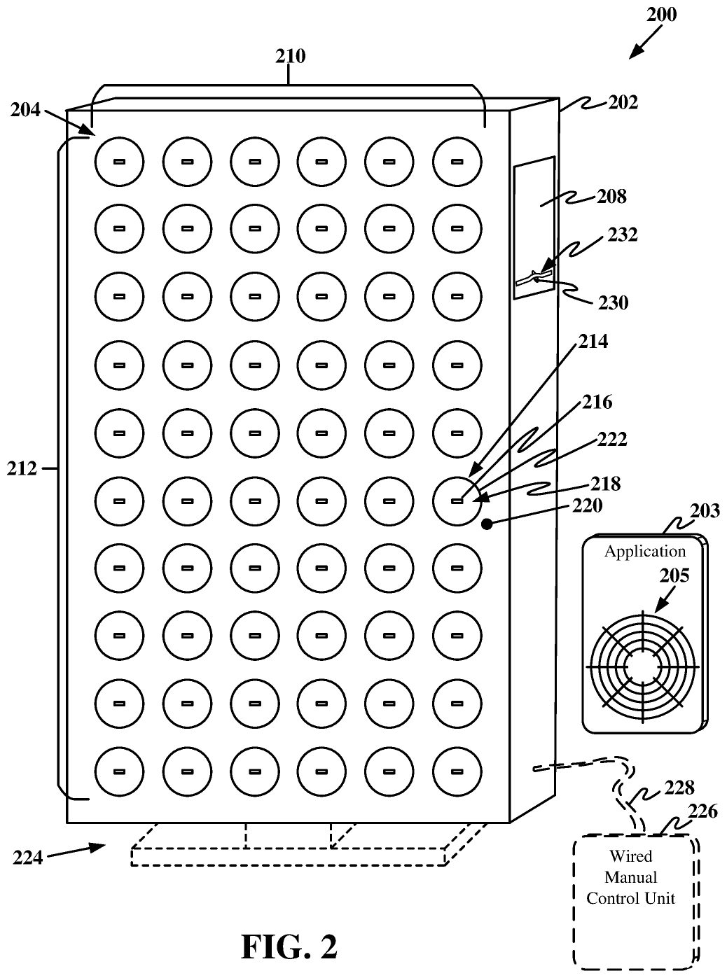

FIG. 2 is an isometric front-right view of an exemplary photobiomodulation system 200 according to some aspects of the disclosure. Included in the exemplary photobiomodulation system 200 are a photobiomodulation device 202 having a light emitting array 204 and a wireless communication device 203 (e.g., a cellular phone, a tablet, a laptop, etc.). The photobiomodulation device 202 may be similar to the photobiomodulation device 100 as shown and described in connection with FIG. 1.

The wireless communication device 203 may be used as a user interface, facilitating remote control of the photobiomodulation device 202 by a user. The wireless communication device 203 may run an application. The application may, among other things, utilize graphics 205 to program and or depict a pattern of illumination of the light emitting array 204. One non-limiting example of such graphics 205 is illustrated as being displayed on a screen of the exemplary wireless communication device 203 depicted in FIG. 2. In the exemplary graphics 205, each of eight radiating spokes depict a respective set of LEDs and/or low level laser emitters. As shown in the non-limiting example, the graphics 205 depict a polar display generated by the application. In general, the graphics 205 may be used to depict which light or set of lights of the light emitting array 204 is illuminated. In one example, the graphics 205 may depict the amount of time left for a given respective set of lights to remain in an on-state based on a length of a line radiating from the center of the graphics 205. The graphics 205 are exemplary and non-limiting. Other shapes, with or without radiating spokes, with fewer or greater than eight radiating spokes, and/or with other features representative of the state of any one or more of the lights of the light emitting array 204 are within the scope of the disclosure.

The wireless communication device 203 is only one example of a device that may be used to interface with the photobiomodulation device 202. According to another example, a wired manual control unit 226, such as a keyboard, keypad, or gaming-controller (examples not explicitly shown) may be used as a user interface. The wired manual control unit 226 may be operationally coupled to the photobiomodulation device 202 by a cable 228. In some examples, the cable 228 may be fixed to (e.g., not removable from) either or both of the photobiomodulation device 202 and the wired manual control unit 226. In other examples, the cable 228 may be removably connected to either or both of the photobiomodulation device 202 and/or the wired manual control unit 226 using connectors. Connectors may include, but are not limited to USB, RJ11, and/or RJ45 types of connectors. In some examples, the photobiomodulation device 202 may operate as a stand-alone unit, rather than as a photobiomodulation system 200 as shown in FIG. 2.

According to one aspect the photobiomodulation device 202 may not need an external user interface (e.g., a wireless communication device 203 or a wired manual control unit 226). In such an aspect, the photobiomodulation device 202 may be controlled from an integrated control panel 208. In the example of FIG. 2, the integrated control panel 208 includes a switch 230 covered by a tape 232. The switch may, for example, provide a user with an ability to turn on or off any UV light emitters in the light emitting array 204. In one example, the photobiomodulation device 202 may be shipped with the switch 230 in a state such that all elements of the light emitting array 204 that may produce UV light are off (i.e., a UV-off state). In one example, the tape 232 may cover the switch 230 to prevent the switch 230 from being switched from a UV-off state to a UV-on state. To change the state of the switch 230, a user would need to physically remove or break the tape 232. Removal of, or breaking of, the tape 232 may represent the consent of, or an acknowledgement of, a user to permit the photobiomodulation device 202 to energize the elements of the light emitting array 204 that may produce UV light. The operation of the switch 230 may be independent of the operation of any other switch, feature, or mechanism that may be used to energize the photobiomodulation device 202.

The photobiomodulation device 202 of FIG. 2 includes a light emitting array 204 having a plurality of columns 210 arranged in a plurality of rows 212. In the example of FIG. 2, there are six columns 210 and ten rows 212, resulting in a light emitting array 204 of sixty elements. A sixty element array having six columns 210 and ten rows 212 is presented as a non-limiting example. Any array of elements having any number of rows and any number of columns is within the scope of the disclosure. By way of example and not limitation, a representative element 214 is identified at the intersection of the sixth column and the sixth row. The representative element 214 includes an LED 216 set into a depression 218 in a front surface 220 of the photobiomodulation device 202. The depression 218 may have an upper outer edge 222 at the level of the front surface 220 (represented by a circle in the illustration of FIG. 2) and a central lower inner surface (relative to the upper outer edge 222) in or upon which the LED 216 is located. The configuration of the elements of the photobiomodulation device 202 (i.e., the arrangement of rows and columns at right angles to each other) of FIG. 2 is presented for purposes of explanation and not limitation. Other configurations of elements (e.g., curved, ellipsoidal, circular, etc.) are within the scope of the disclosure. Furthermore, use of an LED as a light source is exemplary and non-limiting. A low level laser emitter may be used instead of or in addition to an LED. In some examples, one or more of the elements (e.g., LEDs and/or low level laser emitters) of the light emitting array 204 may be removably replaceable. For example, each light source may be connectorized, using, for example, a screw-type or post-type electrical connector/base.

According to some aspects, the depression 218 may form a reflector (e.g., having a parabolic or semi-circular shape). The depression 218 may have a reflective coating (e.g., a mirror coating) but depressions without reflective coatings are within the scope of the disclosure. The depression 218 (coated or uncoated) may aid in directing, focusing, and/or collimating the light emitted from a respective element (e.g., from the LED 216). According to some aspects, the depression 218 may be omitted; the front surface 220 of the photobiomodulation device 202 may be flat (not shown) and the elements (such as LED 216) may sit on, at the same level as, or below, the front surface 220.

In examples where the front surface 220 of the photobiomodulation device 202 either includes depressions (e.g., such as depression 218) or is flat (not shown), respective lenses (not shown) may be placed in a spaced apart configuration from respective elements (such as LED 216). The lenses (not shown) may be formed as one array of a plurality of lenses, having a plurality of respective lens centers that substantially align with a corresponding plurality of element centers. Alternatively, the lenses (not shown) may be individual lenses, where each individual lens corresponds to and is mounted adjacent to one of the plurality of elements (such as LED 216). In some examples, a lens (not shown) associated with an element may aid in directing, focusing, or collimating the light emitted from the element. By way of example and not limitation, each element may have an integrated optical lens included therewith. Other optical devices (e.g., diffusers, collimators, polarizers, filters, etc.) may be configured with respect to the elements of the photobiomodulation device 202 to adjust the emitted light from the elements of the photobiomodulation device 202. Such other optical devices are within the scope of the disclosure.

According to some aspects, the photobiomodulation device 202 may provide data-driven, and personalized photobiomodulation therapeutic treatment to a user (e.g., a patient). The photobiomodulation device 202 may be relatively easy to maintain over time at least because of the long life of LED light sources (such as LED 216) and/or low level laser emitters. According to some aspects, the photobiomodulation device 202 may improve upon known photobiomodulation devices in terms of the components used and the mechanical configuration of the photobiomodulation device 202. According to some examples, the photobiomodulation device 202 may be easy to install on wall-mount and arm-mount mounting systems because of a new use of a standardized threaded hole mounting configuration pattern (not shown). The threaded hole mounting configuration pattern may be controlled by a threaded hole mounting configuration standard, such as the Video Electronics Standards Association (VESA) Interface Standard. The VESA Interface Standard defines the distance in millimeters between four mounting holes on a device. Although VESA is a standard used for televisions and video monitors, the photobiomodulation devices described herein (such as photobiomodulation device 202) may adopt the standard to facilitate use of wall-mount and arm-mount mounting systems marketed for televisions and monitors, for example. In the example of FIG. 2, the photobiomodulation device 202 is depicted with a stand 224. In some examples, the stand 224 may be shipped with the photobiomodulation device but may be removed in favor of mounting the photobiomodulation device 202 to a wall or movable arm.

According to some aspects, the photobiomodulation device 202 may administer photobiomodulation therapy to users by, at least, configuring one or more elements of a light emitting array (such as light emitting array 204) to illuminate according to a set of instructions. The set of instructions may define at least two of: a wavelength, duration of a pulse (e.g., expressed as a duration of on-time, or as a ratio of on-time to off-time, also known as a duty cycle), an intensity, a pattern of repetition of illumination, a number of repetitions of a given pattern of illumination, or a channel. The set of instructions may be designed to obtain a specific health outcome or may be designed to obtain other outcomes (e.g., plant growth, sleep, disinfection, etc.). The set of instructions may also be referred to as a recipe or a protocol herein.

According to some aspects, two or more photobiomodulation devices, such as photobiomodulation device 202, may be used together, and respective controllers (e.g., similar to controller 102 as shown and described in connection with FIG. 1) of each of the two or more photobiomodulation devices may be operationally coupled to one another (e.g., chained together). In this usage case, control signals from the multiple controllers may be passed from one photobiomodulation device to another photobiomodulation device through the physical cable or wirelessly (e.g., over-the-air using any wireless communication standard). The two or more photobiomodulation devices may operate independently or may be interdependent.

In some examples, the light sources of the light emitting array 204 may be fed in parallel. This may avoid a “string of Christmas lights” type failure, where one defective light source extinguishes all light sources in the string of lights because all the light sources in the string of lights were fed in series. Even in cases where respective groups of light sources in the light emitting array 204 are fed from respective drivers, the failure of one driver will only result in the failure of one group of light sources—not all the light sources of the light emitting array 204. Alternatively, the light sources of the light emitting array 204 may be fed in series.

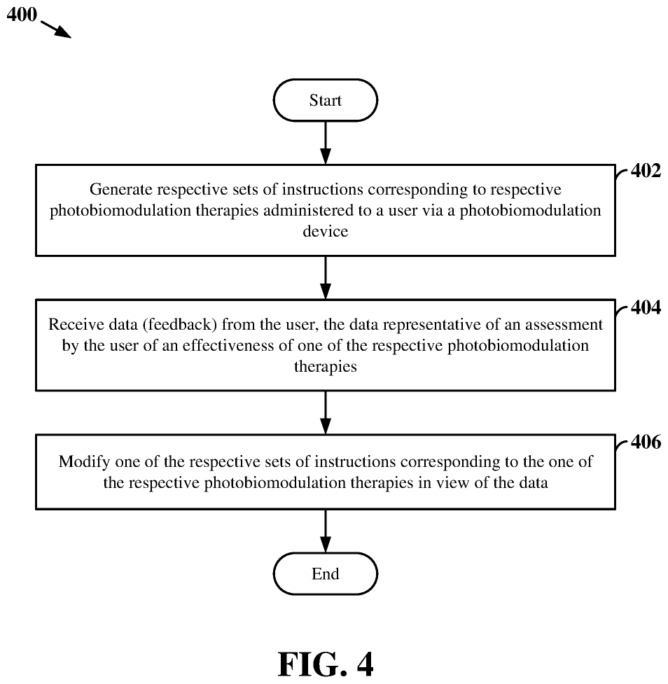

FIG. 4 is a flow chart illustrating one example of an overall process that may be used with a photobiomodulation device according to some aspects of the disclosure. At block 402, an entity may generate respective sets of instructions corresponding to respective photobiomodulation therapies that may be administered to a biological creature (e.g., a person or a plant) via the photobiomodulation device. The entity may be a human entity or may be a machine (e.g., a computer), driven, for example, with machine learning and/or artificial intelligence. In some examples, the respective sets of instructions may be derived based on a synthesis of information gleaned from, for example, scholarly articles, white papers, results of studies of photobiomodulation therapy, and/or feedback of users of photobiomodulation devices. In some examples, the respective sets of instructions may be derived based on preference or learning through use.

By way of example, a set of instructions may be generated that have a result of helping a user of the photobiomodulation device sleep better at night, control mitochondria, promote hair growth, promote weight loss, reduce menstrual cramps, etc. The preceding list is exemplary and non-limiting. The set of instructions may cause a photobiomodulation device to turn on and off multiple wavelengths of light in a predetermined pattern at one or more intensities over a predetermined amount of time. The specific combination of wavelengths, pattern, on/off times, and/or intensities may be tailored according to several factors such as, for example, age and gender; however, additional, or alternative factors may also be utilized to tailor the specific combination of wavelengths, on/off times, and/or intensities. A user may notice an improved sleep pattern by causing the photobiomodulation device to execute the set of instructions (and thereby apply the light to the user in a predetermined pattern) before the user goes to sleep at night.

As indicated above, and by way of example and not limitation, the specific set of instructions may be synthesized from data obtained from scholarly articles, white papers, results of studies of photobiomodulation therapy, and/or from feedback provided by a plurality of respective users of respective photobiomodulation devices having the same or similar demographics as that of the user. By way of example, the data may indicate that a given pattern(s) of a combination of wavelengths, on/off times, and intensities of light, for a total duration of ten minutes, on average, tends to help 50-year-old men obtain improved sleep when the photobiomodulation device is caused to configure a light emitting array (similar to the light emitting array 104 and/or 204 as shown and described in connection with FIG. 1 and/or FIG. 2, respectively) to illuminate light sources according to the specific set of instructions prior to the user's bedtime (e.g., within the hour prior to the user's bedtime). Accordingly, a user (e.g., the exemplified 50-year-old man) using the photobiomodulation device to expose the user to light emitted from the light emitting array according to the specific set of instructions prior to bedtime may receive the benefit of improved sleep.

At block 404, the entity may receive data (e.g., in the way of feedback) from the user, the data representative of an assessment by the user of an effectiveness of one of the respective photobiomodulation therapies. Accordingly, and continuing with the example used above, to determine the effectiveness of a given set of instructions, the photobiomodulation device (or an application associated with the photobiomodulation device) may request feedback from the user in the morning following the treatment. For example, in the case of the 50-year-old man, using the photobiomodulation device according to the set of instructions as described above, upon arising the day following the administration of the photobiomodulation therapy, the user may be asked (e.g., by displaying a question for the user on a display screen) if the user (i.e., the 50-year-old-man) obtained the desired improvement in sleep. According to some aspects, the answer provided by the user may be a binary answer; for example, the user may reply that his sleep last night was better than his sleep the night before or worse than his sleep the night before. According to another aspect, the answer may be provided by the user using a scale; for example, the user may reply that on a scale of 1 to 10 with one representing worst and 10 representing best, the sleep last night rated a 7 (e.g., possibly meaning the sleep was somewhat better than the night before but not the best hoped for). The preceding is only one example. In another example, the photobiomodulation device (or an application associated with the photobiomodulation device) may remind the user to do a session. In another example, the photobiomodulation device (or an application associated with the photobiomodulation device) may later request feedback on actual outcome versus intended outcome. In still another example, the photobiomodulation device (or an application associated with the photobiomodulation device) may suggest other therapies based on demographics or on used treatments or both.

At block 406, the entity may modify one of the respective sets of instructions corresponding to the one of the respective photobiomodulation therapies in view of the data. Continuing with the preceding example, the entity may obtain the answer (i.e., the data, the feedback) from the user and, based on the answer, may determine that an increase in the total duration of treatment (e.g., from 10 minutes to 12 minutes) might improve the outcome for the user. The photobiomodulation device may indicate (e.g., by displaying a statement on a display screen) that increasing the duration of treatment from 10 minutes to 12 minutes may result in a better outcome. The photobiomodulation device may then ask (e.g., by displaying a question on a display screen) if the user would like to increase the duration of the treatment to the recommended 12 minutes. The user may respond by selecting Yes or No, or by entering the user's own choice for the increased duration (e.g., 14 minutes). Thereafter, the photobiomodulation device may store the new duration in association with the given set of instructions. In the evening of the day following the first administration of photobiomodulation therapy, the photobiomodulation device, when caused by the user to execute the given set of instructions, may execute the given set of instructions using the new total duration. The process of obtaining feedback and adjusting total duration (or any other adjustable factor) may continue indefinitely or may continue until the user instructs the photobiomodulation device to end the feedback aspect of the treatment.

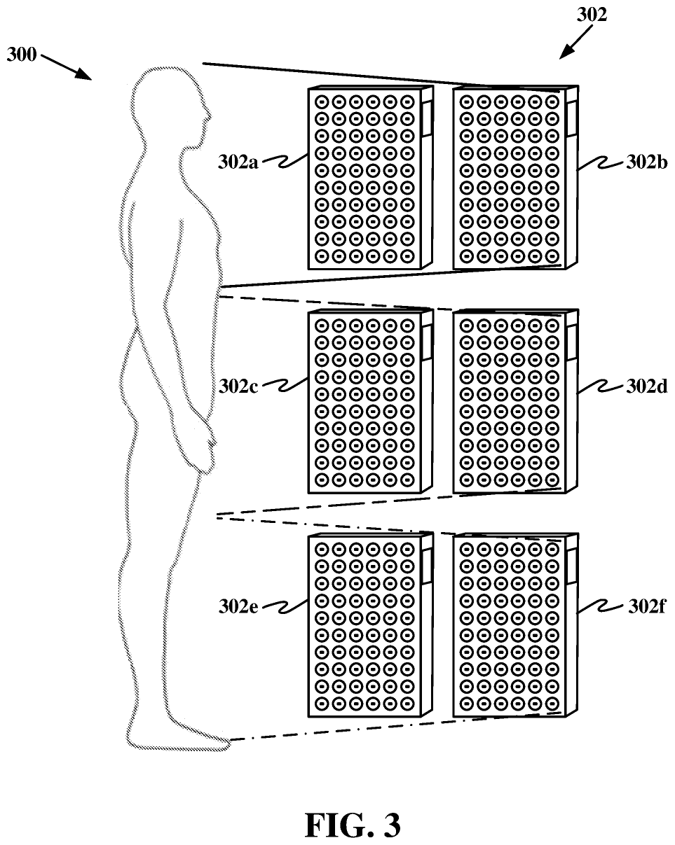

FIG. 3 is a conceptualized schematic view of a person 300 standing in front of, and receiving photobiomodulation therapy from, a representative array of photobiomodulation devices 302. Each of the photobiomodulation devices 302a, 302b, 302c, 302d, 302e, 302f of the representative array of photobiomodulation devices 302 may be similar to the photobiomodulation devices 200 of FIG. 2, according to some aspects of the disclosure. In the example of FIG. 3, the array of photobiomodulation devices 302 is represented as a two wide by three tall array of individual photobiomodulation devices. The representative array of photobiomodulation devices 302 is exemplary and non-limiting. A greater or fewer number of individual photobiomodulation devices may be utilized. Additionally, the array of photobiomodulation devices need not be restricted to a two by three array. Any size, order, configuration, assemblage, or collection of photobiomodulation devices, each in any orientation, is within the scope of the disclosure. In one example of the array of photobiomodulation devices 302 of FIG. 3, each of the photobiomodulation devices 302a, 302b, 302c, 302d, 302e, 302f may be mounted to a wall (not shown) via a VESA type mount or other type of mount. In another example of the array of photobiomodulation devices 302 of FIG. 3, each of the photobiomodulation devices 302a, 302b, 302c, 302d, 302e, 302f may be mounted to a stand or rack (not shown) fixedly mounted to or movably resting on a floor (not shown). The preceding examples are non-limiting. Other ways and structures may be used to support the photobiomodulation devices 302 of FIG. 3 without limitation.

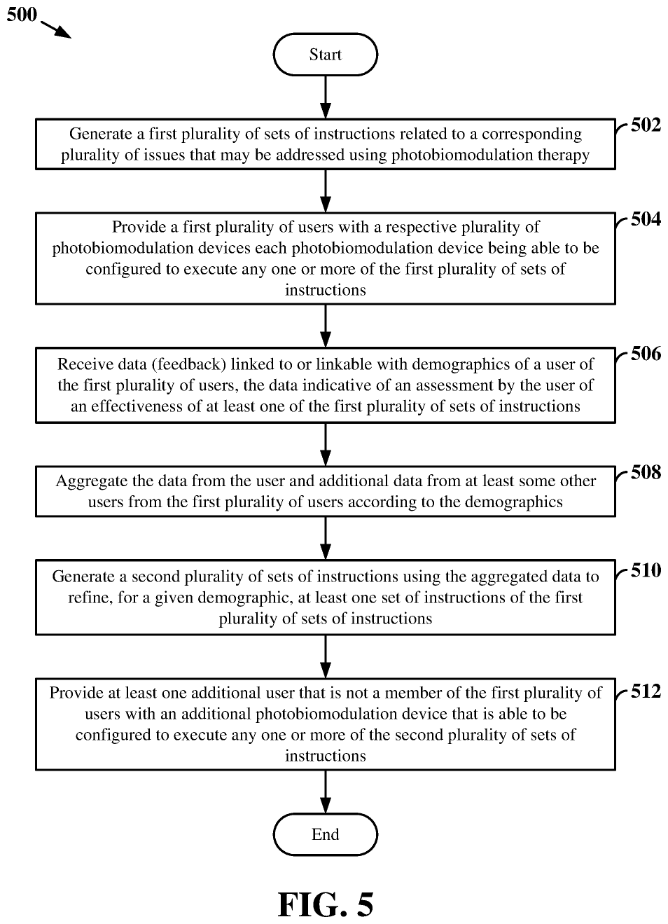

FIG. 5 is a flow chart depicting a process associated with a photobiomodulation device according to some aspects of the disclosure. At block 502, an entity may generate a first plurality of sets of instructions related to a corresponding plurality of issues that may be addressed using photobiomodulation therapy. Block 502 is similar to block 402 as shown and described in connection with FIG. 4, the details of which will not be repeated for the sale of brevity.

At block 504, a first plurality of users may be provisioned with a respective plurality of photobiomodulation devices each photobiomodulation device being able to be configured to execute any one or more of the first plurality of sets of instructions.

At block 506, a server may receive data (e.g., feedback) linked to or linkable with demographics of a user of the first plurality of users, the data representative of an assessment by the user of an effectiveness of at least one of the first plurality of sets of instructions. Block 506 is similar to block 404 as shown and described in connection with FIG. 4, the details of which will not be repeated for the sale of brevity.

At block 508, the server may aggregate the data from the user and additional data from at least some other users from the first plurality of users according to the demographics. The server may aggregate the data in association with the demographics of a plurality of users to determine, on average, for a given demographic, each parameter controlled by the set of instructions.

At block 510, the server may generate a second plurality of sets of instructions using the aggregated data to refine, for a given demographic, at least one set of instructions of the first plurality of sets of instructions.

At block 512, the entity may provide at least one additional user that is not a member of the first plurality of users (e.g., a new user) with an additional photobiomodulation device (a new photobiomodulation device belonging to the new user) that is able to be configured to execute any one or more of the second plurality of sets of instructions

By way of example and not limitation with respect to blocks 508, 510, and 512, the server may aggregate a total duration of treatment for the set of instructions related to improvements in sleep. The average of the aggregated values may be employed in sets of instructions utilized for new users of photobiomodulation device. For example, when a new user first uses the new user's photobiomodulation device, the set of instructions related to improvements in sleep may be tailored, ahead of the first administration of the set of instructions for the new user, to the duration found on average (from the aggregated total) for the new user's demographic. Of course, the new user, via the new user's photobiomodulation device (and/or application associated therewith), may provide feedback on the efficacy of the set of instructions related to improvements in sleep, and may store the new user's selected total duration for the benefit of the new user. That is, the new user need not accept the total duration first associated with the new user's photobiomodulation device but may adjust the total duration according to the new user's feedback and preferences.

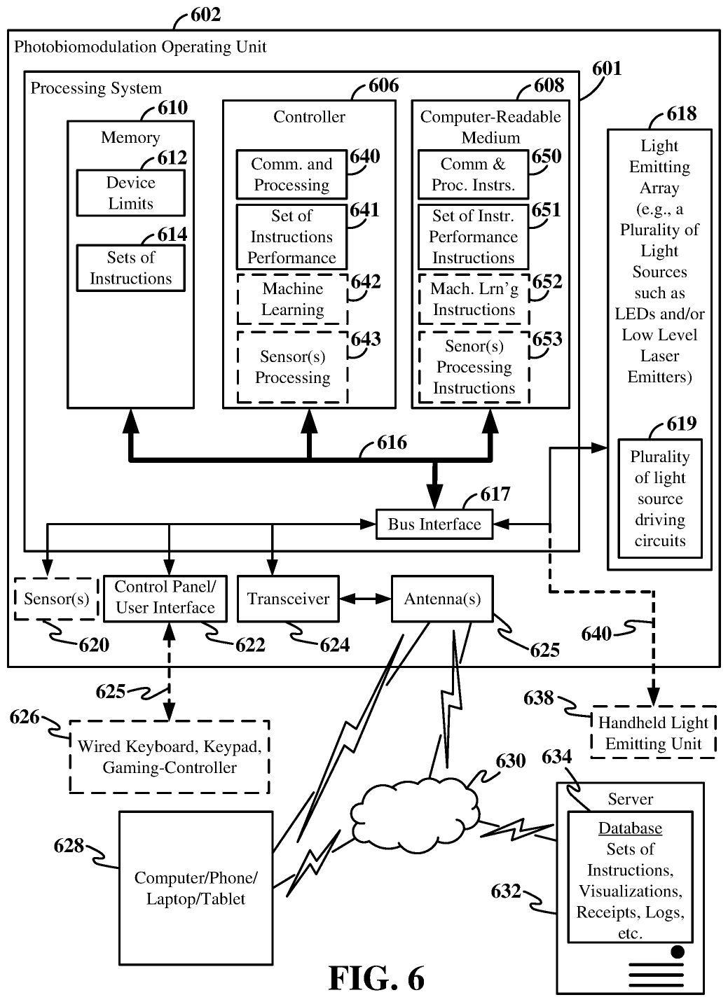

FIG. 6 is a block diagram illustrating an example of a hardware implementation of a photobiomodulation device 600 employing a processing system 601 according to some aspects of the disclosure. For example, the photobiomodulation device 600 may correspond to the photobiomodulation device 100 as shown and described in connection with FIG. 1 and/or the photobiomodulation device 202 as shown and described in connection with FIG. 2.

In accordance with various aspects of the disclosure, an element, or any portion of an element, or any combination of elements, may be implemented with a processing system 601 that includes one or more controllers (or processors), such as controller 606. Examples of controller 606 include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionality described throughout this disclosure. In various examples, the photobiomodulation device 600 may be configured to perform any one or more of the functions described herein. The controller 606, as utilized in the photobiomodulation device 600, may be used to implement any one or more of the methods or processes described and illustrated, for example, in FIGS. 4, 5, 7, 8, 9, and/or 10.

In this example, the processing system 601 may be implemented with a bus architecture, generally represented by the bus 616. The bus 616 may include any number of interconnecting buses and bridges depending on the specific application of the processing system 601 and the overall design constraints. The bus 616 links together various circuits, including one or more controllers (represented generally by the controller 606), a memory 610, and computer-readable media (represented generally by the computer-readable medium 608). The bus 616 may also link various other circuits such as timing sources, peripherals, voltage regulators, and power management circuits, which are well known in the art and will not be described further.

A bus interface 617 provides an interface between the bus 616 and a plurality of additional circuits/functions, including a light emitting array 618 (e.g., an LED panel) (similar to the light emitting array 104 as shown and described in connection with FIG. 1 and/or the light emitting array 204 as shown and described in connection with FIG. 2), a plurality of light source driving circuits 619 associated with the light emitting array 618 (e.g., a plurality of LED driver circuits), optional sensor(s) 620, a control panel/user interface 622, a transceiver 624, and antenna(s) 625. The preceding lists were exemplary and non-limiting. Additionally, or alternatively, a handheld light emitting unit 638 may also include a plurality of light sources such as LEDs and/or low level laser emitters. The handheld light emitting unit 638 may be operationally coupled to the photobiomodulation operating unit 602 by a cable 640. In some examples, the cable 640 may be fixed to (e.g., not removable from) either or both of the photobiomodulation operating unit 602 and the handheld light emitting unit 638. In other examples, the cable 640 may be removably connected to either or both of the photobiomodulation operating unit 602 and/or the handheld light emitting unit 638 using connectors. Connectors may include, but are not limited to USB, RJ11, and/or RJ45 types of connectors.

The transceiver 624 and antenna(s) 625 may provide a communication interface or a means for communicating with various other apparatus over a transmission medium (e.g., an air interface). The control panel/user interface 622 may provide a user with a way to directly interface with the photobiomodulation device 600. The control panel/user interface 622 may be operationally coupled to a wired keyboard, keypad, or gaming-controller 626 (any of which may include a remote display, a touch screen, a speaker, a microphone, etc.), which may provide optional ways for a user to interface with the photobiomodulation device 600 according to some aspects described herein. The wired keyboard, keypad, or gaming controller may be operationally coupled to the control panel/user interface 622 by a cable 625. In some examples, the cable 625 may be fixed to (e.g., not removable from) either or both of the photobiomodulation operating unit 602 and the wired keyboard, keypad, or gaming-controller 626. In other examples, the cable 625 may be removably connected to either or both of the photobiomodulation operating unit 602 and/or the wired keyboard, keypad, or gaming-controller 626 using connectors. Connectors may include, but are not limited to USB, RJ11, and/or RJ45 types of connectors.

The controller 606 may be responsible for managing the bus 616 and general processing, including executing software stored on the computer-readable medium 608. The software, when executed by the controller 606, causes the processing system 601 to perform the various functions described below for any particular apparatus. The computer-readable medium 608 and the memory 610 may also be used for storing data manipulated by the controller 606 when executing software. For example, the memory 610 may store device limits 612 and sets of instructions 614 generated to provide a photobiomodulation therapy that obtains a specific health outcome. The preceding list is exemplary and non-limiting.

One or more controllers (such as the controller 606) in the processing system 601 may execute software. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. The software may reside on the computer-readable medium 608.

The computer-readable medium 608 may be a non-transitory computer-readable medium. A non-transitory computer-readable medium includes, by way of example, a magnetic storage device (e.g., hard disk, floppy disk, magnetic strip), an optical disk (e.g., a compact disc (CD), or a digital versatile disc (DVD)), a smart card, a flash memory device (e.g., a card, a stick, or a key drive), a random access memory (RAM), a read-only memory (ROM), a programmable ROM (PROM), an erasable PROM (EPROM), an electrically erasable PROM (EEPROM), a register, a removable disk, and any other suitable medium for storing software and/or instructions that may be accessed and read by a computer. The computer-readable medium 608 may reside in the processing system 601, external to the processing system 601, or distributed across multiple entities, including the processing system 601. The computer-readable medium 608 may be embodied in a computer program product. By way of example, a computer program product may include a computer-readable medium in packaging materials. In some examples, the computer-readable medium 608 may be part of the memory 610. Those skilled in the art will recognize how best to implement the described functionality presented throughout this disclosure depending on the particular application and the overall design constraints imposed on the overall system. In some examples, the computer-readable medium 608 may be implemented on an article of manufacture, which may further include one or more other elements or circuits, such as the controller 606 and/or memory 610.

In some aspects, the photobiomodulation device 600 may include the memory 610 and the controller 606 coupled to the memory, the controller 606 and the memory 805, at least, may be configured to perform any of the methods, functions, or algorithms described herein. In some aspects of the disclosure, the controller 606 may include circuitry configured for various functions. For example, the controller 606 may include communication and processing circuitry/function 640 (also referred to as the communication and processing circuitry 640 for the sake of brevity), configured to communicate with other devices internal and external to the photobiomodulation device 600, for example, via interfaces therebetween.

In some examples, the communication and processing circuitry 640 may include one or more hardware components that provide the physical structure that performs processes related to communication (e.g., signal reception and/or signal transmission) and signal processing (e.g., processing a received signal and/or processing a signal for transmission). For example, the communication and processing circuitry 640 may include one or more modems. In some examples, the communication and processing circuitry 640 may include one or more hardware components that provide the physical structure that performs processes related to processing, such as, for example, obtaining device limits 612 and sets of instructions 614 from the memory 610 and processing such data to limit the features of the photobiomodulation device 600 accordingly. In some implementations where the communication involves receiving data from sensor(s) 620, for example, the communication and processing circuitry 640 may obtain the data, process the data, and output the processed data. For example, the communication and processing circuitry 640 may output the processed data to another component of the controller 606, the memory 610, or the bus interface 617. In some examples, the communication and processing circuitry 640 may receive one or more of signals, messages, other information, or any combination thereof. In some examples, the communication and processing circuitry 640 may include functionality for a means for receiving and/or a means for transmitting.

In some aspects of the disclosure, the controller 606 may include a set of instructions performance circuitry/function 641 configured for various functions, including defining at least two of: a wavelength of illumination, a duration of illumination, an intensity of illumination, a pattern of illumination, or a number of repetitions of the pattern of illumination associated with one or more light sources of a plurality of light sources included with the light emitting array 618. For example, the set of instructions performance circuitry/function 641 may be utilized to interface with the plurality of light source driving circuits 619, where each of the plurality of light sources is driven by one of the plurality of light source driving circuits 619. In some aspects, the set of instructions performance circuitry/function 641 may be utilized to interface with the plurality of light source driving circuits 619, where the plurality of light sources is divided into a plurality of subgroups and each of the plurality of subgroups is driven by one of the plurality of light source driving circuits 619, for example. In some other aspects, one or more individual light sources of the plurality of light sources may each be respectively driven by one of the plurality of light source driving circuits 619.

Modifying a wavelength of illumination, a duration of illumination, an intensity of illumination, a pattern of illumination, or a number of repetitions of the pattern of illumination associated with each of a plurality of light sources included with the light emitting array 618 may result in a change, for example, a change over time, of the spectrum of light being emitted from the light emitting array 618.

For example, modifying intensity may facilitate a variation (e.g., a variable control) of the light energy produced by, for example, the at least one light source of the plurality of light sources (e.g., at least one LED of a plurality of LEDs). Controlling the intensity of light energy produced (e.g., light emitted), for example, by increasing or decreasing a voltage applied to a light source may provide for a changing the spectrum of light emitted from the light emitting array 618. In other words, the change of spectrum need not be restricted to a change of wavelength (i.e., a change of color) but may include a change of any of the parameters associated with any light source of the plurality of light sources of the light emitting array 618, including but not limited to a change to the presence, duration, intensity, color, etc. of the light emitted from the light emitting array 618.

Modification of the duration of the light emitted from the at least one light source of the plurality of light sources of the light emitting array 618 may relate to modification of an on-time and an off-time (e.g., one cycle), modification of a duty cycle that controls the ratio between on-time and off-time, or modification of any other aspect that may affect the total duration of illumination from any one or more of the plurality of light sources of the light emitting array 618. Modification of a presence of a light emitted from at least one light source of the plurality of light sources may include modification to the on/off state of any light source of the plurality of light sources of the light emitting array 618.

The set of instructions performance circuitry 641 may further be configured to execute set of instructions performance instructions 651 (e.g., software) stored on the computer-readable medium 608 to implement one or more functions described herein.

In some aspects of the disclosure, the controller 606 may include a machine learning circuitry/function 642 configured for various functions, including, for example, learning and adapting without following explicit instructions, by using algorithms and statistical models to analyze and draw inferences from patterns in data. The data may be received from the sensor(s) 624, for example, or received from the communication and processing circuitry/function 640 described above. The machine learning circuitry/function 642 may further be configured to execute machine learning instructions 652 (e.g., software) stored on the computer-readable medium 608 to implement one or more functions described herein.

In some aspects of the disclosure, the controller 606 may include, for example, sensor(s) processing circuitry/function 643, configured for various functions including, for example, obtaining information from sensor(s) 620 and acting on such information. For example, sensor(s) 620 may include a temperature sensor (e.g., a thermometer or a thermocouple), a light meter that senses (e.g., measures) light intensity reflected from the skin of a user (e.g., a patient receiving phototherapy), a distance measuring sensor, etc. The preceding list is exemplary and not limiting; other sensor(s) are within the scope of the disclosure.

For example, the sensor(s) 620 may include a color measuring device configured to measure skin coloration. The sensor(s) processing circuitry/function 643 may use the data from the color measuring device to account for skin coloration changes caused by photobiomodulation treatment and may, for example, modify a set of instructions to cause any adjustments needed in view of the change to skin coloration. For example, the sensor(s) 620 may include a pulse monitor to monitor heart rate. The sensor(s) processing circuitry/function 643 may use the measured heart rate to determine the efficacy of the photobiomodulation therapy (e.g., a photobiomodulation therapy designed to lower heart rates) and modify a set of instructions accordingly. For example, the sensor(s) 620 may include a breath sensor to monitor breathing. The sensor(s) processing circuitry/function 643 may use the data from the breath sensor to determine the efficacy of a given photobiomodulation therapy (e.g., a photobiomodulation therapy designed to increase lung capacity) and modify a set of instructions accordingly.

For example, the sensor(s) 620 may include a camera, or a local device, such as a camera of a user's/owner's computer/phone/tablet 628 coupled to the photobiomodulation device. Still, images or video from the camera may be analyzed (e.g., using machine learning models) to determine the spatial position of the user's body parts. This spatial position information may be utilized to adjust the settings of the photobiomodulation device 600 by sending commands, for example, from the sensor(s) processing circuitry/function 643 to other controller 606 circuitry/functions. For example, a particular command may specify a certain light intensity based on a user being one meter from the photobiomodulation device 600; if the user is closer, the controller 606 may decrease the intensity of light emitted from the light emitting array 618; if the user is farther away controller 606 may increase the intensity of light emitted from the light emitting array 618.

According to another aspect, the associated camera image(s) may be analyzed to determine what ambient light is present and adjust the light output of the light emitting array 618 accordingly. For example, if there is a prevalence of blue ambient light, then blue light emitters of the plurality of light sources may be decreased in intensity to maintain a predetermined overall intensity level.

The camera image(s) may also be utilized to determine the user's skin phenotype. Several scales exist, such as Google's Monk Skin Tone scale, to classify skin levels for light processing. This camera sensor information may be used to determine which wavelengths of light are easily absorbed by the current user. The controller 606 may then adjust the intensity, for example, to amplify or dampen particular wavelengths accordingly.

If the camera can produce image data containing infrared or ultraviolet wavelengths, this sensor information may be utilized to adjust the light output of the photobiomodulation therapeutic unit 604. For example, if the user is reflecting a large amount of infrared during photobiomodulation treatment then the infrared output may be increased accordingly or vice versa. For example, if the user is not reflecting ultraviolet light while ultraviolet light is being emitted, then ultraviolet light may be decreased to maintain safe levels or vice versa. These levels may be based on current research and general, principled anecdotes regarding how different skin phenotypes react to natural and synthetic light systems (e.g., sunburning).

Camera imaging may also be used to determine what types of photobiomodulation therapy may be utilized based on the user's mood or persona. Several machine learning models exist that can analyze a human face and determine the user's disposition based on metrics in facial measurements. For example, if the user's mood is determined to be non-energetic or poor then a prescription (or recipe) that may be known to, or thought to, increase energy may be activated.

User imaging may also be used to authenticate a user and thus, load their preferences and programs based on the visual recognition. For example, a user may look at the camera, and the controller may identify them if they are previously registered and load their desired photobiomodulation therapy prescription(s) (or recipe(s)) based on the user's mood, time-of-day, etc.

Camera imaging may also be used to determine what types of photobiomodulation therapy may be utilized based on skin condition. Machine learning models can determine skin health based on blood flow and color, especially when comparing current imaging to a user's past images. Some models may reasonably approximate the user's blood pressure and heart rate. For example, if a user is desirous of a photobiomodulation therapy that induces rest, but their condition shows unrest, then light element parameters may be automatically (without user intervention) adjusted by the controller 606 accordingly.

These adjustments may be made on the user level. These adjustments may also be made on the system level by allowing machine learning to promote appropriate modifications to global sets of instructions. These adjustments may also be made automatically based on known desired outcomes.

The sensor(s) processing circuitry/function 643 may further be configured to execute sensor(s) processing instructions 653 to implement one or more functions described herein.

According to some aspects, the photobiomodulation device 600 (or the controller 606) described herein may communicatively couple to a server 632, which may be a remote server and may pull data from and push data to a database 634 maintained at the server 632. Coupling may be via a communications network, represented by a cloud 630 in FIG. 6. Use of data, such as sets of instructions, prescriptions, visualizations, receipts, logs, etc. stored in the database 634 at the server 632 may facilitate individualization, reusability, and continuous improvement of the photobiomodulation devices described herein. Data storage on a server may also facilitate gathering data pertinent to individual users or groups of users sharing a common demographic. The gathered data may be used to build individual (personal) or group profiles and set individual (personal) or group goals. Data storage on the server 632 may also facilitate the aggregation of feedback comparing actual outcomes to the goals of photobiomodulation therapy (e.g., compare outcomes to intent).

A cloud computing system, for example, a system that utilizes the server 632, may enable broad storage of anonymized usage data. This serves to drive a custom intelligence platform for developing and selecting optimal sets of instructions associated with photobiomodulation therapies, in-depth reporting per user of their own usage data, and a medium through which medical professionals can incorporate photobiomodulation data into their analysis of users' outcomes. This usage data may include a timestamp of each usage, which set of instructions was implemented (whether the photobiomodulation therapy was modified; e.g., ended early, dimmed, etc.), the efficacy of each implementation, and geo-location, for example.

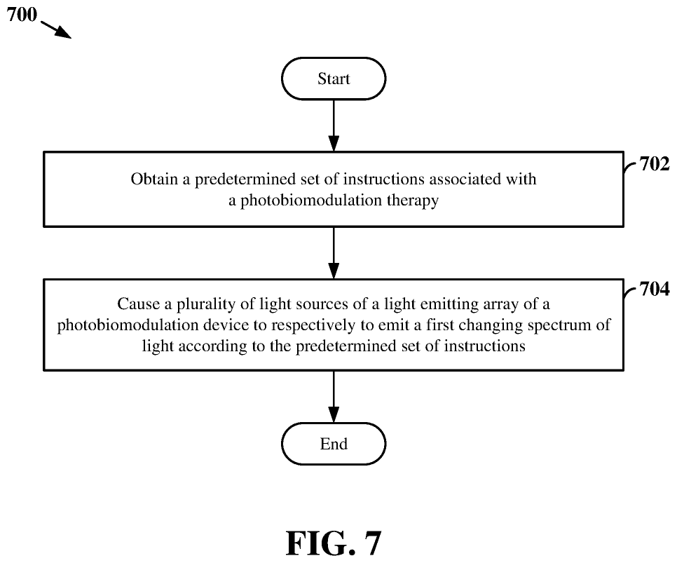

FIG. 7 is a flow chart illustrating an example of a process 700 (e.g., a method) of photobiomodulation therapy at a photobiomodulation device according to some aspects of the disclosure. As described below, some or all illustrated features may be omitted in a particular implementation within the scope of the present disclosure, and some illustrated features may not be required to implement all examples. In some examples, the process 700 may be carried out by the photobiomodulation device 100 of FIG. 1, 202 of FIG. 2, or 600 of FIG. 6. In some examples, the process 700 may be carried out by any suitable apparatus or means for carrying out the functions or algorithm described below.

The process 700 may include, at block 702, obtaining, for example by a controller such as controller 606 as shown and described in connection with FIG. 6, of a set of instructions (e.g., a predetermined set of instructions) associated with a photobiomodulation therapy. At block 704, the controller may cause a plurality of light sources of a light emitting array of a photobiomodulation device to respectively to emit a first changing spectrum of light according to the set of instructions.

In some examples, the set of instructions may be generated to provide a photobiomodulation therapy that obtains a specific health outcome or may be generated to obtain other outcomes (e.g., plant growth, sleep, disinfection, etc.). In some examples, the photobiomodulation device may include a plurality of light source driving circuits, where each of the plurality of light sources is driven by one of the plurality of light source driving circuits. In some examples, the photobiomodulation device may include a plurality of light source driving circuits, where the plurality of light sources is divided into a plurality of subgroups and each of the plurality of subgroups is driven by one of the plurality of light source driving circuits. In some examples, each of the plurality of light sources is at least one of a light emitting diode or a low level laser emitter.

According to some aspects, the set of instructions may define at least two of: a wavelength of illumination, a duration of illumination, an intensity of illumination, a pattern of illumination, or a number of repetitions of the pattern of illumination associated with each of the plurality of light sources.

According to some examples, the controller of the photobiomodulation device may be configured to receive data representative of an assessment of an effectiveness, as a photobiomodulation therapy designed to obtain a specific health outcome (or another outcome such as improved plant growth, improved sleep, disinfection of surfaces, etc.), of the first changing spectrum of light emitted according to the set of instructions. Of course, a measure of improvement of an outcome resulting from application of photobiomodulation therapy may be measured relative to the outcome obtained before application of the photobiomodulation therapy. In some examples, the controller may be configured to determine, based on the data, a modification to the set of instructions to increase the effectiveness of the first changing spectrum of light emitted, output a message offering an opportunity to accept the modification, and store in a memory of the photobiomodulation device, in response to receiving an input indicative of an acceptance of the modification, a modified set of instructions in place of the set of instructions. Still further, in some examples, the controller may also be configured to cause the plurality of light sources of the light emitting array to respectively emit a second changing spectrum of light according to the modified set of instructions. By way of example, the emission of the second changing spectrum of light might occur on a day following the day during which the photobiomodulation device emitted the set of instructions (e.g., before the set of instructions was modified).

According to some examples, an application associated with the photobiomodulation device may be configured to: receive data representative of an assessment of an effectiveness, as a photobiomodulation therapy designed to obtain a specific health outcome, of the first changing spectrum of light emitted according to the set of instructions; and transmit the data to a server associated with the photobiomodulation device.

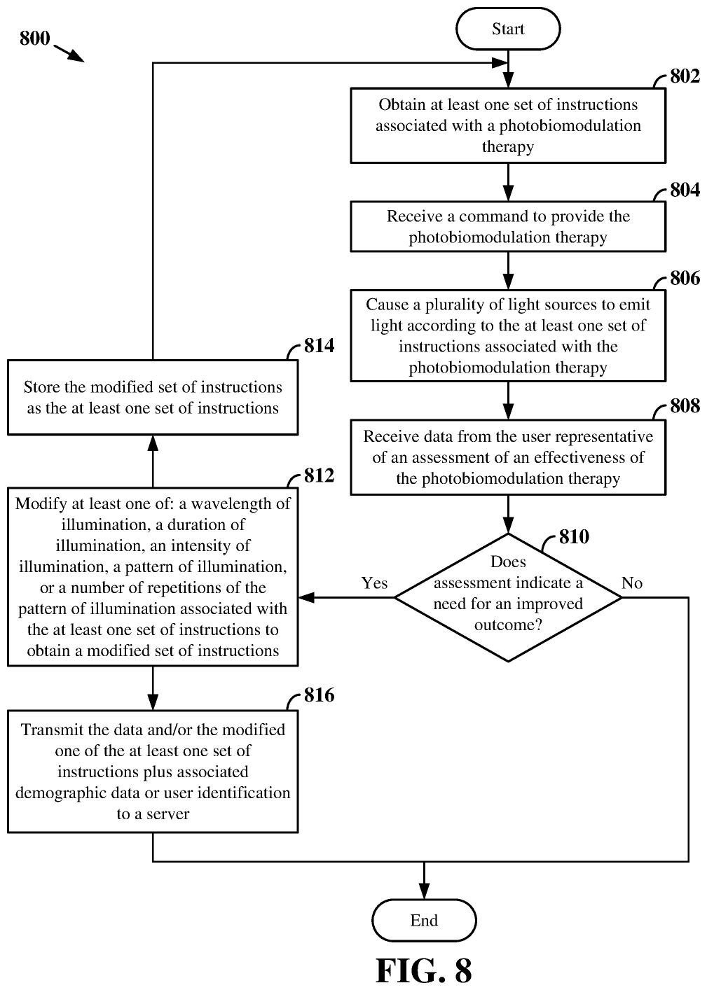

FIG. 8 is a flow chart illustrating an example of a process 800 (e.g., a method) of updating set of instructions of a user at both a photobiomodulation device and at a server associated with the photobiomodulation device according to some aspects of the disclosure. As described below, some or all illustrated features may be omitted in a particular implementation within the scope of the present disclosure, and some illustrated features may not be required to implement all examples. In some examples, the process 900 may be carried out by the photobiomodulation device 100 of FIG. 1, 202 of FIG. 2, or 600 of FIG. 6. In some examples, the process 900 may be carried out by any suitable apparatus or means for carrying out the functions or algorithm described below.

At 802, the photobiomodulation device may obtain at least one set of instructions associated with a photobiomodulation therapy. Sets of instructions have been exemplified above and their description will not be repeated for the sake of brevity. At 804, the photobiomodulation device may receive a command to provide the photobiomodulation therapy. According to some aspects, the command may be received as an input from a user of the photobiomodulation device. At 806, in response to receiving the command at 804, the photobiomodulation device, or a controller thereof, may cause a plurality of light sources to emit light according to the at least one set of instructions associated with the photobiomodulation therapy. As mentioned, sets of instructions have been exemplified above and their description will not be repeated for the sake of brevity.

At 808, following the photobiomodulation therapy, the photobiomodulation device, or a controller thereof, may receive data from the user representative of an assessment of an effectiveness of the photobiomodulation therapy.

At 810, if the assessment does not indicate a need for an improved outcome (e.g., the user's assessment indicated that the photobiomodulation therapy provided the desired outcome), then no change is needed to the set of instructions and the process 800 may end. However, if the assessment indicated a need for an improved outcome (e.g., the user's assessment indicated that the photobiomodulation therapy did not provide the desired outcome), then, at 812, the photobiomodulation device, or a controller thereof, may modify at least one of: a wavelength of illumination, a duration of illumination, an intensity of illumination, a pattern of illumination, or a number of repetitions of the pattern of illumination associated with the at least one set of instructions to obtain a modified set of instructions.

From block 812, the process 800 may advance to both block 814 and 816 in parallel. At block 814 the photobiomodulation device, or a controller thereof, may store the modified set of instructions as the at least one set of instructions (e.g., may overwrite the at least one set of instructions with the modified set of instructions) and the process 800 may return to block 802. Thereafter, by obtaining the at least one set of instructions at block 802, the photobiomodulation device, or a controller thereof, is, in fact, obtaining the modified set of instructions. At block 816 the photobiomodulation device, or a controller thereof, may transmit the data and/or the modified one of the at least one set of instructions plus associated demographic data or user identification to a server, such as server 632 as shown and described in connection with FIG. 6.

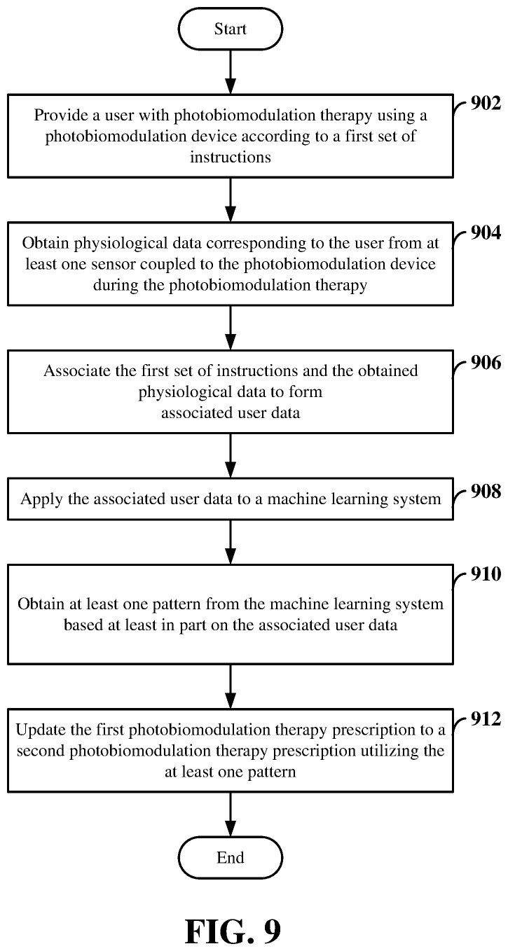

FIG. 9 is a flow chart illustrating an example of a process 900 (e.g., a method) associated with machine learning according to some aspects of the disclosure. As described below, some or all illustrated features may be omitted in a particular implementation within the scope of the present disclosure, and some illustrated features may not be required to implement all examples. In some examples, the process 900 may be carried out by the photobiomodulation device 100 of FIG. 1, 202 of FIG. 2, or 600 of FIG. 6. In some examples, the process 900 may be carried out by any suitable apparatus or means for carrying out the functions or algorithm described below.

At 902, the photobiomodulation device may provide a user with photobiomodulation therapy using the photobiomodulation device (e.g., using light emitted from a light emitting array 618 as shown and described in connection with FIG. 6) according to a first set of instructions. The first set of instructions may be stored in and retrieved from a memory, such as the memory 610 as shown and described in connection with FIG. 6. For example, the light emitting array 618, in conjunction with set of instructions performance circuitry 641, as shown and described in connection with FIG. 6, may provide a means for providing a user with photobiomodulation therapy using the photobiomodulation device according to a first set of instructions.

At 904, the photobiomodulation device may obtain physiological data corresponding to the user from at least one sensor coupled to the photobiomodulation device during the photobiomodulation therapy. For example, the communication and processing circuitry/function 640, in conjunction with the sensor(s) 620, and/or the sensor(s) processing circuitry/function 643, may provide a means for obtaining physiological data corresponding to the user from at least one sensor coupled to the photobiomodulation device during the photobiomodulation therapy.

At 906, the photobiomodulation device may associate the first set of instructions and the obtained physiological data to form associated user data. For example, the communications and processing circuitry/function 640, as shown and described in connection with FIG. 6, may provide a means for associating the first set of instructions and the obtained physiological data to form associated user data.

At 908, the photobiomodulation device may apply the associated user data to a machine learning system. For example, the machine learning circuitry/function 642, as shown and described in connection with FIG. 6, may provide a means for applying the associated user data to a machine learning system.

At 910, the photobiomodulation device may obtain at least one pattern from the machine learning system, based at least in part on the associated user data. According to some aspects, the machine learning system extracted the at least one pattern from a plurality of photobiomodulation-related data, including the associated user data. For example, the communication and processing circuitry/function 640 and/or the machine learning circuitry/function 642, both as shown and described in connection with FIG. 6, may provide a means for obtaining at least one pattern from the machine learning system, based at least in part on the associated user data.

At 912, the photobiomodulation device may update the first set of instructions to a second set of instructions utilizing the at least one pattern. For example, the communication and processing circuitry/function 640, as shown and described in connection with FIG. 6, may provide a means for updating the first set of instructions to a second set of instructions utilizing the at least one pattern. The updating may include updating a, such as the database 634 at the server 632, according to aspects herein.

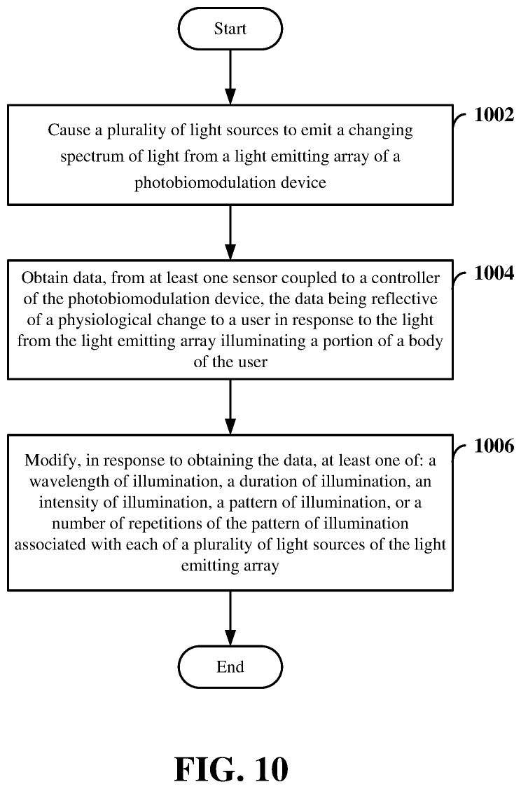

FIG. 10 is a flow chart illustrating an example of a process 1000 (e.g., a method) of modifying parameters associated with an emitting of a spectrum of light in response to changes in physiological data of a user according to some aspects of the disclosure. As described below, some or all illustrated features may be omitted in a particular implementation within the scope of the present disclosure, and some illustrated features may not be required to implement all examples. In some examples, the process 1000 may be carried out by the photobiomodulation device 100 of FIG. 1, 202 of FIG. 2, or 600 of FIG. 6. In some examples, the process 1000 may be carried out by any suitable apparatus or means for carrying out the functions or algorithm described below.

At 1002, the photobiomodulation device may cause a plurality of light sources to emit a changing spectrum of light from a light emitting array of a photobiomodulation device. According to some aspects, the photobiomodulation device may obtain a set of instructions and may cause the plurality of light sources to emit the changing spectrum of light according to the set of instructions. In some examples, the set of instructions may be stored in a memory of the photobiomodulation device or at a server remotely coupled to the photobiomodulation device.

At 1004, the photobiomodulation device may obtain data, from at least one sensor coupled to a controller of the photobiomodulation device, the data being reflective of a physiological change to a user in response to the light from light emitting array illuminating a portion of a body of the user. In some examples, the at least one sensor may be a camera, and the photobiomodulation device (or a controller thereof) may be further configured to obtain, without user intervention, the set of instructions on a basis of photo recognition of the user utilizing an image of the user captured by the camera. For example, the sensor(s) processing circuitry/function 643, as shown and described in connection with FIG. 6, may provide a means for obtaining data from at least one sensor coupled to a controller of the photobiomodulation device, the data being reflective of a physiological change to a user in response to the light from the light emitting array illuminating a portion of a body of the user.

At 1006, the photobiomodulation device may modify, in response to obtaining the data, at least one of: a wavelength of illumination, a duration of illumination, an intensity of illumination, a pattern of illumination, or a number of repetitions of the pattern of illumination associated with each of a plurality of light sources of the light emitting array. For example, the set of instructions performance circuitry/function 641, as shown and described in connection with FIG. 6, may provide a means for modifying, in response to obtaining the data, at least one of: a wavelength of illumination, a duration of illumination, an intensity of illumination, a pattern of illumination, or a number of repetitions of the pattern of illumination associated with each of a plurality of light sources of the light emitting array

### Human-Machine Integration

The controller may utilize a human-machine integration to operate devices, such as those exemplified herein. According to one aspect, the controller may be configured to activate and deactivate one or more photobiomodulation devices and execute user-specific sets of instructions (e.g., recipes, protocols) to facilitate a general health protocol that incorporates one or more photobiomodulation devices. The controller may interface with the one or more photobiomodulation devices, a database, and a server, to receive input from and provide output to a photobiomodulation device user and/or the server.

As used herein, sets of instructions may include commands to: turn on or off each wavelength of light; turn on or off different sections of a light emitting array (e.g., top-left quadrant, bottom half, etc.); set a duration after which a series of lighting elements may be de-energized, set the intensity (e.g., the brightness) of a particular wavelength (or frequency) of light, of the emitted light, or any combination thereof. According to one aspect, the setting of the intensity may employ Pulse-Width Modulation (PWM), which is a method of cycling the amount of time power is applied to a given lighting element (e.g., setting the duty cycle).

The controller may be configured to collect data passively or actively from one or more photobiomodulation devices. The one or more photobiomodulation devices may be connected by a standard communication protocol (e.g., Bluetooth or Universal Serial Bus (USB)); they may additionally or alternatively use a proprietary communication protocol. The collected data can be tied to a particular user.

According to some aspects, the controller may be configured to interact with health-related services such as biohacking, medical, and therapeutic services. The controller may be capable of interfacing with a variety of systems under the direction of the user.

Current Internet of Things (IoT) technology may include custom hardware that communicates with custom cloud software through a custom Application Programming Interface. Many systems recreate this entire stack of sub-systems for each product. Typically, the data is reserved for the originator's use alone, and the user is allowed to see a subset of it or a generated report. Many valid platforms for health-related services do not provide the capability for users to store, track, and disseminate their data.

However, the photobiomodulation devices exemplified herein may provide for the integration of devices. They may also allow the aggregation of their data from disparate devices and provide the aggregated data to consumers (such as doctors, coaches, etc.). The photobiomodulation devices exemplified herein may integrate a user with the user's data even when the user does not own the photobiomodulation device. The storage of data obtained in connection with the use of the photobiomodulation devices exemplified herein is consistent with existing privacy regulations with the intent of being capable of interfacing with medical systems.

In one example, a platform that may be used to operate the photobiomodulation devices exemplified herein may be a Hardware-Interface Layer (HIL), data collection Application Programming Interface (API), a User-Experience (UX) system, a Recipe system, and 3rd Party Integrations.

The HIL is a collection of known, connectable devices and their capabilities. The HIL includes a control scheme for each known device, allowing it to interact with a user interface.

The API is a data service that is accessible through standard Internet interfaces (HTTPS, WSS, etc.) of a central server and/or via distributed, cryptographic peer-to-peer mechanisms such as InterPlanetary File System (IPFS) or blockchain.

The UX system provides standard mechanisms for retrieving and displaying data to an end-user. This includes a subset of the API specifically for users, owners, or consumers. This also includes purpose-built libraries to display elements and interact with the API on supported platforms.

The Recipe system allows owners to create and prescribe programs of operations “recipes” (also referred to herein as sets of instructions or protocols) for particular devices and particular users. A recipe may also be a particular program used by many devices.

3rd Party Integrations give the ability to interchange data with other systems for particular users. The user and the proprietor control consumer access. These integrations conform to necessary regulations and meet required design guidelines for our chosen realm.

Using a cloud computing infrastructure may allow device owners to view usage history of their devices. In this capacity, the controller may allow device owners to provide the usage of devices as a service to registered users. The device owner may then see anonymized usage of their photobiomodulation device(s), such as which sets of instructions were run and times and dates of usage. A cloud computing infrastructure may also allow a device user to see their usage metrics, as described previously, regardless of whether they own the photobiomodulation device.

A cloud computing system may also quickly analyze user feedback and, thus, adjust sets of instructions. A user may receive a short questionnaire regarding a recent session of photobiomodulation therapy. The user's response may be analyzed and used to adjust future, similar durations and power levels (e.g., intensities) for the given user, and, according to some aspects, for other users as well.

A local computer or mobile device may be used to offer a user experience that may be visually stimulating. A local computer or mobile device may provide a more efficient interface to accept user input and provide a richer, more meaningful display to the user. A local computer or mobile device may store recipes (sets of instructions) at the local computer or the mobile device, respectively.

The local computer may authorize access to attached devices when no cloud/internet access is available. It may store multiple user profiles, which include enough data about a user to allow the user to use an attached photobiomodulation device or devices in a manner they are accustomed to without interruption due to lack of connectivity.

The local computer may also store data fragments corresponding to users authorized to use an attached device. This data may be stored locally, indefinitely, until such time as it can be synchronized with the cloud/remote data storage, which is more permanent and more widely accessible.

The local computer also allows a convenient, constantly available interface for users to update their personal information and goals related to the photobiomodulation device. This information can be stored and utilized by a central system or the local system to develop an intelligent framework of prescriptions (or recipes) unique to the user to attain the user's particular goals.

The local computer may also connect to multiple photobiomodulation devices and operate all of them independently or in a coordinated manner. For example, if two photobiomodulation devices are positioned such that one is in front of a user and one is behind a user, the local computer can determine which way the user is facing and adjust the front and rear photobiomodulation device parameters accordingly such that each runs a different sequence of commands.

The controller may accept commands over standard communication channels like USB serial and Bluetooth Low Energy (BLE) Generic ATTribute Profile (GATT) functionality. An example command structure follows:
Channel0_dutyCycle=58
Channel3_frequency=730

These commands and the sequences of commands are drawn from the global cloud data storage system. The local computer (e.g., a mobile phone, tablet, etc.) may determine what type of photobiomodulation device is attached and read available commands and sequences from the global system. These commands are then sent to the controller in the prescribed manner.

The local computer may simultaneously emit these commands to USB and Bluetooth interfaces. It may be configured to disable one or the other of these interfaces. The controller may also be configured to disable one or the other of these interfaces.

The controller may also emit data for status updates and command responses. An example of such a command response follows:
Channel3_frequency˜730
Distance˜15.3

The controller may also make use of a distance sensor. An example of this is a hypersonic range finder (e.g., a sonar). This distance measurement may be utilized to adjust the maximum intensity of the lighting element or may be fed to the local computing device and utilized by the local computing device to adjust and/or give finer usage details.

The controller may also emit commands to one or more USB serial connections to allow a leader/follower or daisy-chained control topology. This would enable several small devices to be incorporated into a larger fixture and operated as one. The nature of the controller is such that whatever commands are received by the controller, by any means, wired or wireless, are forwarded through wired or wireless outputs. For example, the main device's controller receives a command to activate a certain wavelength of light at a certain frequency at a certain brightness; the controller will execute the command and then output the same command to a USB serial port and to a BLE mesh network such that one subsidiary, the hard-wired device is activated, and a subsidiary, wirelessly tethered device is activated, via a single command. In this manner, any number of devices can be controlled by connecting only to the first device. The arrangement of connections between devices does not matter.

The controller may also implement several “hard-wired” safety features. Because the controller may be able to adjust the power levels of some or all lighting elements by their position or their characteristics (e.g., wavelength). The controller may be commanded to activate, deactivate, energize, or de-energize based on certain rules. For example, if the local computing device determines the user's eye level at 1.5 m, it may signal the controller to deactivate all near-infrared lighting elements at such a height that would directly impact the eyes. For example, if UV is deemed to have a maximum exposure time of 8 minutes, then the controller may be signaled to de-energize all UV after 8 minutes.

The controller, with or without a local computing device, may determine that no user is present based on a distance sensor or other sensors such as visual imagery and deactivate after a prescribed time. For example, if nothing is detected by the distance sensor and any programming or artificial intelligence built into the photobiomodulation device does not detect a human form, all light emission may be ended (turned off).

The photobiomodulation device may incorporate a highly modular design such that enclosures, power supplies, lighting, and control system elements are discrete and changeable.

Power supplies may separate from the main enclosure so they can be hidden from view or mounted elsewhere to reduce heat accumulation. Connection to the photobiomodulation device may then be made using standard wiring schemes and connectors known to the electrical community.

Enclosures may be purpose fit with interlocking capabilities, extending the daisy-chaining concept's usability. Enclosures may also incorporate standard mounting schemes like VESA, allowing the device owner/operator to use standard mounting rigs and/or surfaces.

Individual lighting elements in the photobiomodulation therapeutic application unit may be interchangeable and independent. This allows each lighting element to contain multiple emitters which operate at different wavelengths. Additionally, these lighting elements may be individually removed and replaced without affecting the operation of other elements in the photobiomodulation therapeutic application unit.

According to some aspects, a main controller board may be a distinct sub-assembly that may be mounted alongside the photobiomodulation device. For example, it may be mounted in the main lighting system enclosure. For example, it may be mounted in the power supply enclosure. It may have a display for direct user operation and/or necessary information such as the current settings or addressing information detailing how to connect with the device. For example, it may display a Quick Response (QR) code that gives the controlling application enough information to connect. This displayed QR code may change as needed to provide different information. For example, it may include connection status so the local computer can verify the connection.

Photobiomodulation therapy utilizing the exemplary photobiomodulation devices described herein may allow users to be as engaged with the product as they desire. This increases the usefulness of the photobiomodulation device and improves the user's outcomes. With the available technology outlined herein, users can interact with a photobiomodulation device with no effort: based on existing programming and non-touch feedback signaling their presence. Users can also interact more deeply, or on a lower level, with the photobiomodulation device if desired (and permitted). Users may also have a more native experience with high-level functionality (e.g., prescriptions or recipes—sequences of commands run in series or parallel) at the photobiomodulation device at the time of usage.

Not only does this enlighten and engage users in their usage of the photobiomodulation device, but it also incorporates that usage into users' communities, allows a deeper understanding of paths to their desired outcomes, and facilitates the help of others while incorporating photobiomodulation therapies and applications into their overall health plan.

While certain examples and features have been described and shown in the accompanying drawings, it is to be understood that such examples and features are merely illustrative of and not restrictive on the broad invention and that this invention is not limited to the specific constructions and arrangements shown and described, since various other modifications may occur to those ordinarily skilled in the art.

Within the present disclosure, the word “exemplary” is used to mean “serving as an example, instance, or illustration.” Any implementation or aspect described herein as “exemplary” is not necessarily to be construed as preferred or advantageous over other aspects of the disclosure. Likewise, the term “aspects” does not require that all aspects of the disclosure include the discussed feature, advantage, or mode of operation. The term “coupled” is used herein to refer to the direct or indirect coupling between two objects. For example, if object A physically touches object B, and object B touches object C, then objects A and C may still be considered coupled to one another—even if they do not directly physically touch each other. For instance, a first object may be coupled to a second object even though the first object is never directly physically in contact with the second object. The terms “circuit” and “circuitry” are used broadly, and intended to include both hardware implementations of electrical devices and conductors that, when connected and configured, enable the performance of the functions described in the present disclosure, without limitation as to the type of electronic circuits, as well as software implementations of information and instructions that, when executed by a processor, enable the performance of the functions described in the present disclosure.

One or more of the components, steps, features and/or functions illustrated in FIGS. 1-10 may be rearranged and/or combined into a single component, step, feature, or function or embodied in several components, steps, or functions. Additional elements, components, steps, and/or functions may also be added without departing from novel features disclosed herein. The apparatus, devices, and/or components illustrated in FIGS. 1-10 may be configured to perform one or more of the methods, features, or steps described herein. The novel algorithms described herein may also be efficiently implemented in software and/or embedded in hardware.

It is to be understood that the specific order or hierarchy of steps in the methods disclosed is an illustration of exemplary processes. Based upon design preferences, it is understood that the specific order or hierarchy of steps in the methods may be rearranged. The accompanying method claims present elements of the various steps in a sample order and are not meant to be limited to the specific order or hierarchy presented unless specifically recited therein.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” Unless specifically stated otherwise, the term “some” refers to one or more. A phrase referring to “at least one of” a list of items refers to any combination of those items, including single members. As an example, “at least one of: a, b, or c” is intended to cover: a; b; c; a and b; a and c; b and c; and a, b and c. The construct A and/or B is intended to cover: A; B; and A and B. The word “obtain” as used herein may mean, for example, acquire, calculate, construct, derive, determine, receive, and/or retrieve. The preceding list is exemplary and not limiting. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

What is claimed is:

1. A photobiomodulation device, comprising:
a memory;
a light emitting array; and
a controller coupled to the memory and the light emitting array, wherein the controller is configured to:
cause a plurality of light sources of the light emitting array to respectively emit a first changing spectrum of light according to a set of instructions stored in the memory.

2. The photobiomodulation device of claim 1, wherein the set of instructions is generated to provide a photobiomodulation therapy that obtains a specific health outcome.

3. The photobiomodulation device of claim 1, further comprising:
a plurality of light source driving circuits, wherein each of the plurality of light sources is driven by one of the plurality of light source driving circuits.

4. The photobiomodulation device of claim 1, further comprising:
a plurality of light source driving circuits, wherein the plurality of light sources is divided into a plurality of subgroups and each of the plurality of subgroups is driven by one of the plurality of light source driving circuits.

5. The photobiomodulation device of claim 1, wherein each of the plurality of light sources is at least one of a light emitting diode or a low level laser emitter.

6. The photobiomodulation device of claim 1, wherein the set of instructions define at least two of: a wavelength of illumination, a duration of illumination, an intensity of illumination, a pattern of illumination, or a number of repetitions of the pattern of illumination associated with one or more light sources of the plurality of light sources.

7. The photobiomodulation device of claim 1, wherein the controller is further configured to:
receive data representative of an assessment of an effectiveness of the first changing spectrum of light emitted according to the set of instructions as a photobiomodulation therapy designed to obtain a specific health outcome.

8. The photobiomodulation device of claim 7, where the controller is further configured to:
determine, based on the data, a modification to the set of instructions to increase the effectiveness of the first changing spectrum of light emitted;
output a message offering an opportunity to accept the modification; and
store in the memory, in response to receiving an input indicative of an acceptance of the modification, a modified set of instructions in place of the set of instructions.

9. The photobiomodulation device of claim 8, wherein the controller is further configured to cause the plurality of light sources of the light emitting array to respectively emit a second changing spectrum of light according to the modified set of instructions.

10. The photobiomodulation device of claim 1, wherein an application associated with the photobiomodulation device is configured to:
receive data representative of an assessment of an effectiveness, as a photobiomodulation therapy designed to obtain a specific health outcome, of the first changing spectrum of light emitted according to the set of instructions; and
transmit the data to a server associated with the photobiomodulation device.

11. A method operational at a photobiomodulation device, comprising:
causing a plurality of light sources of a light emitting array of the photobiomodulation device to respectively emit a first changing spectrum of light according to a set of instructions.

12. The method of claim 11, wherein the set of instructions is generated to provide a photobiomodulation therapy that obtains a specific health outcome.

13. The method of claim 11, further comprising:
driving each of the plurality of light sources by a respective one of a plurality of light source driving circuits according to the set of instructions.

14. The method of claim 11, wherein the plurality of light sources is divided into a plurality of subgroups, the method further comprising:
driving each of the plurality of subgroups by a respective one of a plurality of light source driving circuits according to the set of instructions.

15. The method of claim 11, wherein each of the plurality of light sources is at least one of a light emitting diode or a low level laser emitter.

16. The method of claim 11, wherein the set of instructions define at least two of: a wavelength of illumination, a duration of illumination, an intensity of illumination, a pattern of illumination, or a number of repetitions of the pattern of illumination associated with one or more light sources of the plurality of light sources.

17. The method of claim 11, further comprising:
receiving data representative of an assessment of an effectiveness of the first changing spectrum of light emitted according to the set of instructions as a photobiomodulation therapy designed to obtain a specific health outcome.

18. The method of claim 17, further comprising:
determining, based on the data, a modification to the set of instructions to increase the effectiveness of the first changing spectrum of light emitted;
outputting a message offering an opportunity to accept the modification; and
storing, in response to receiving an input indicative of an acceptance of the modification, a modified set of instructions in place of the set of instructions.

19. The method of claim 18, further comprising:
causing the plurality of light sources of the light emitting array of the photobiomodulation device to respectively emit a second changing spectrum of light according to the modified set of instructions.

20. The method of claim 11, further comprising:
receiving, by an application associated with the photobiomodulation device, data representative of an assessment of an effectiveness, as a photobiomodulation therapy designed to obtain a specific health outcome, of the first changing spectrum of light emitted according to the set of instructions; and
transmitting, by the application, the data to a server associated with the photobiomodulation device.
